(19) United States
(12) Patent Application Publication
(10) Pub. No.: US 2026/0207245 A1
(43) Pub. Date: Jul. 23, 2026

(54) PULSED FIELD ABLATION SYSTEM AND LEAKAGE PROTECTION THEREOF

(71) Applicant: Medtronic, Inc., Minneapolis, MN (US)

(72) Inventors: Steven J. Fraasch, Maple Grove, MN (US); Vinicius Binotti, Elk River, MN (US); Jon E. Zimmer, Shoreview, MN (US); Qin Zhang, Shoreview, MN (US); Thomas A. Weber, Woodbury, MN (US)

(21) Appl. No.: 19/138,622

(22) PCT Filed: Nov. 17, 2023
(86) PCT No.: PCT/IB2023/061648

§ 371 Date: Jun. 12, 2025

Related U.S. Application Data
(60) Provisional application No. 63/387,466, filed on Dec. 14, 2022.

(51) Int. Cl. A61B 18/12; A61B 18/00; A61B 18/14; G01R 19/165; G01R 31/52; H02H 3/16

(52) U.S. Cl. CPC A61B 18/1233; A61B 18/1492; G01R 19/16571; G01R 31/52; H02H 3/16; A61B 2018/00577; A61B 2018/00613; A61B 2018/00833

(57) ABSTRACT

One aspect provides a pulsed field ablation system including a bridge circuit configured to deliver bipolar and biphasic voltage pulses to a catheter. A leakage fault protection circuit is electrically coupled to the bridge circuit and includes a first comparator. An electronic processor is electrically coupled to the bridge circuit and the leakage fault protection circuit. The electronic processor is configured to determine a first offset threshold value to correct a first offset referenced at the first comparator and set a first threshold parameter of the first comparator based on the first offset threshold value and a leakage current parameter of the pulsed field ablation system. The electronic processor is also configured to determine a leakage fault when a detection parameter corresponding to the leakage fault satisfies the first threshold parameter at the first comparator.

This application claims the benefit of U.S. Provisional Patent Application Ser. No. 63/387,466, filed Dec. 14, 2022, the entire content of which is incorporated herein by reference.

### FIELD

The present technology is generally related to leakage fault protection for pulsed field ablation systems.

### BACKGROUND

Pulsed field ablation delivers a sequence of fast, bipolar, and biphasic high voltage pulses to perform irreversible electroporation of tissue. Pulsed field ablation is used to treat, among other things, cardiac arrhythmias and atrial fibrillation. Pulsed field ablation may also be used as an oncology treatment for cancer.

### SUMMARY

A pulsed field ablation system may be used to deliver a sequence of fast, bipolar, and biphasic high voltage direct-current (DC) pulses to a patient such that successful irreversible electroporation occurs. A catheter is used to deliver the high-voltage pulses to the patient. Pulsed field ablation systems use significant power and energy. Several transistor switches, for example, insulated-gate bipolar transistors (IGBTs), field effect transistors (FETs), or the like are used to control the pulse delivery. A leakage fault in the pulsed field ablation system, for example, in the transistor switches, may result in undesirable or non-therapeutic current being delivered to the patient.

Accordingly, there is a need for leakage fault protection in pulsed field ablation systems.

The techniques described herein generally relate to leakage fault protection circuit and method for pulsed field ablation systems. The leakage fault protection circuit and method help to reduce undesirable or non-therapeutic current being delivered to a patient. Additionally, the leakage fault protection circuit and method account for component offsets in the leakage fault protection circuit to help avoid false detection of leakage faults.

One aspect provides a pulsed field ablation system including a bridge circuit configured to deliver bipolar and biphasic voltage pulses to a catheter, a leakage fault protection circuit electrically coupled to the bridge circuit and including a first comparator, and an electronic processor electrically coupled to the bridge circuit and the leakage fault protection circuit. The electronic processor is configured to determine a first offset threshold value to correct a first offset referenced at the first comparator and set a first threshold parameter of the first comparator based on the first offset threshold value and a leakage current parameter of the pulsed field ablation system. The electronic processor is also configured to determine a leakage fault when a detection parameter corresponding to the leakage fault satisfies the first threshold parameter at the first comparator.

Another aspect provides a method for leakage fault protection in a pulsed field ablation system including a bridge circuit configured to deliver bipolar and biphasic voltage pulses to a catheter and a leakage fault protection circuit electrically coupled to the bridge circuit and including a first comparator. The method includes determining, using an electronic processor, a first offset threshold value to correct a first offset referenced at the first comparator and setting, using the electronic processor, a first threshold parameter of the first comparator based on the first offset threshold value and a leakage current parameter of the pulsed field ablation system. The method also includes determining, using the first comparator, a leakage fault when a detection parameter corresponding to the leakage fault satisfies the first threshold parameter at the first comparator.

Various embodiments, examples, aspects, and features are set forth in the description below and the accompanying drawings. Other embodiments, examples, aspects, features, objects, and advantages of the techniques described in this disclosure will be apparent from the description and drawings, and from the claims.

### BRIEF DESCRIPTION OF DRAWINGS

The accompanying figures, where like reference numerals refer to identical or functionally similar elements throughout the separate views, which together with the detailed description below are incorporated in and form part of the specification and serve to further illustrate various embodiments, examples, aspects, and features that include the claimed subject matter, and to explain various principles and advantages of aspects of those embodiments, examples, aspects, and features.

FIG. 1 is a simplified block diagram that illustrates a pulsed field ablation system in accordance with some examples.

FIG. 2 illustrates an example of voltage pulses delivered by the pulsed field ablation system of FIG. 1 in accordance with some examples.

FIG. 3 is a simplified schematic that illustrates a bridge circuit of the pulsed field ablation system of FIG. 1 in accordance with some examples.

FIG. 4 is a simplified block diagram that illustrates a leakage fault protection circuit of the pulsed field ablation system of FIG. 1 in accordance with some examples.

FIG. 5 is a flowchart for a method for determining an offset of the leakage fault protection circuit of FIG. 4 in accordance with some examples.

FIG. 6 is a flowchart for a method for leakage fault detection in the pulsed field ablation system of FIG. 1 in accordance with some examples.

FIG. 7 is a flowchart for a method for leakage fault protection in the pulsed field ablation system of FIG. 1 in accordance with some examples.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of examples of the present invention.

The apparatus and method components have been represented where appropriate by conventional symbols in the drawings, showing only those specific details that are pertinent to understanding the embodiments, examples, aspects, and features so as not to obscure the disclosure with details that will be readily apparent to those of ordinary skill in the art having the benefit of the description herein.

### DETAILED DESCRIPTION

Before any embodiments, examples, aspects, and features are explained in detail, it is to be understood that those embodiments, examples, aspects, and features are not limited in their application to the details of construction and the arrangement of components set forth in the following description or illustrated in the following drawings. Other embodiments, examples, aspects, and features are possible and are capable of being practiced or carried out in various ways.

Also, it is to be understood that the phraseology and terminology used herein is for the purpose of description and should not be regarded as limiting. The terms “mounted,” “connected,” and “coupled” are used broadly and encompass both direct and indirect mounting, connecting, and coupling. The terms “connected” and “coupled” are not restricted to physical or mechanical connections or couplings, and can include electrical connections or couplings, whether direct or indirect. Electronic communications and notifications described herein may be performed using any known or future-developed means including wired connections, wireless connections, etc.

For ease of description, some or all of the example systems presented herein are illustrated with a single exemplar of each of its component parts. Some examples may not describe or illustrate all components of the systems. Other embodiments may include more or fewer of each of the illustrated components, may combine some components, or may include additional or alternative components.

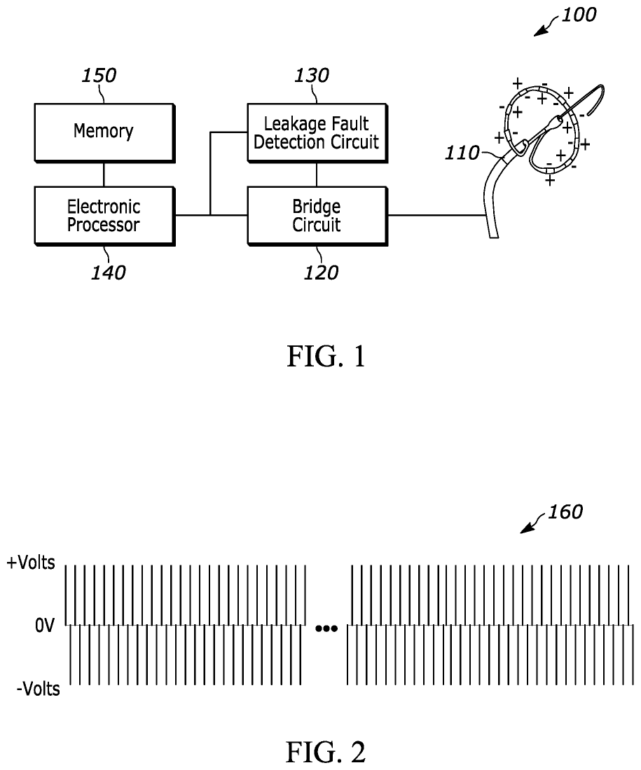

FIG. 1 is a simplified block diagram of an example pulsed field ablation system 100. In the example shown, the pulsed field ablation system 100 is used to deliver a sequence of fast, bipolar, and biphasic voltage pulses 160 (for example, as shown in FIG. 2) to a catheter 110 to perform irreversible electroporation of tissue. The pulsed field ablation system 100 includes a bridge circuit 120, a leakage fault protection circuit 130, an electronic processor 140, and a memory 150.

The catheter 110 is a multi-electrode catheter including a plurality of electrodes arranged successively around an enclosed or semi-enclosed area. The catheter 110 delivers the voltage pulses to tissue within the enclosed or semi-enclosed area. In some examples, the catheter 110 may be a disposable catheter 110 that is disposed after each use, while a new disposable catheter 110 is connected to the pulsed field ablation system 100 for every distinct procedure. The bridge circuit 120 is electrically coupled to the catheter 110. The bridge circuit 120 generates and delivers the voltage pulses to the catheter 110. The leakage fault protection circuit 130 is electrically coupled to the bridge circuit 120. The leakage fault protection circuit 130 detects leakage faults within the bridge circuit 120.

The electronic processor 140 is electrically coupled to the bridge circuit 120 and the leakage fault protection circuit 130 and is configured to control and monitor the bridge circuit 120 and the leakage fault protection circuit 130. In some instances, the electronic processor 140 is implemented as a microprocessor with separate memory, such as the memory 150. In other embodiments, the electronic processor 140 may be implemented as a microcontroller (with memory 150 on the same chip). In other embodiments, the electronic processor 140 may be implemented using multiple processors. In addition, the electronic processor 140 may be implemented partially or entirely as, for example, a field-programmable gate array (FPGA), an applications specific integrated circuit (ASIC), an x86 processor, and the like and the memory 150 may not be needed or be modified accordingly. In the example, illustrated, the memory 150 includes non-transitory, computer readable memory that stores instructions that are received and executed by the electronic processor 140 to carry out the functionality of the pulsed field ablation system 100 described herein. The memory 150 may include, for example, a program storage area and a data storage area. The program storage area and the data storage area may include combinations of different types of memory, such as read-only memory and random-access memory. In some embodiments, the pulsed field ablation system 100 includes one electronic processor 140 and/or a plurality of electronic processors 140 in a computer cluster arrangement, one or more of which may be executing none, all, or a portion of the applications of the pulsed field ablation system 100.

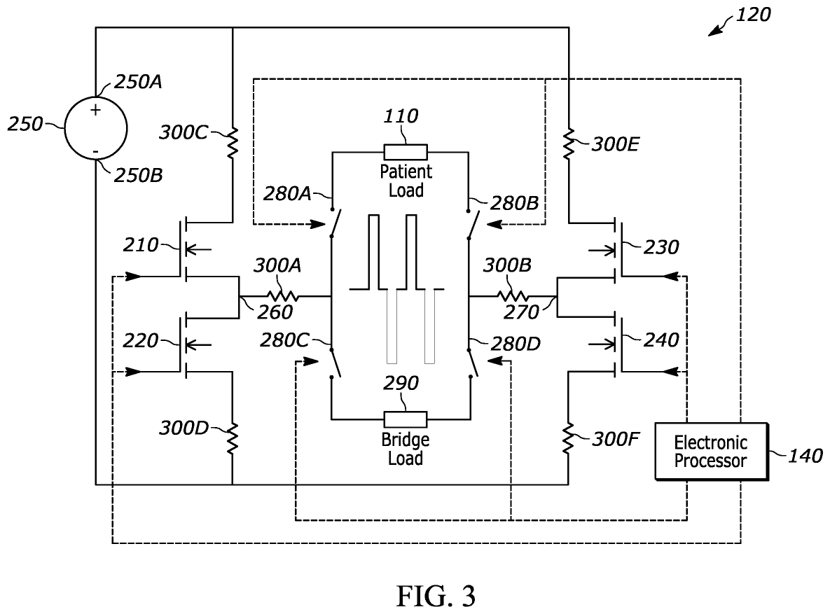

FIG. 3 illustrates a simplified schematic of the bridge circuit 120. In the example illustrated, the bridge circuit 120 is a full H-bridge circuit. In other examples, the bridge circuit 120 may be an inverter bridge circuit, or the like. The full H-bridge circuit is made up of a first transistor switch 210, a second transistor switch 220, a third transistor switch 230, and a fourth transistor switch 240. The transistor switches 210-240 include, for example, insulated-gate bipolar transistors (IGBTs), field effect transistors (FETs), and/or the like.

A power supply 250 provides high-voltage power to the H-bridge circuit. For pulsed field ablation systems 100, the high-voltage power may be in the range of between 300 Volts and 2000 Volts. The power supply 250 generates the high-voltage potential between a positive power supply node 250A and a negative power supply node 250B (for example, electric ground). The power supply 250 may include a high-voltage battery system or an alternating current (AC) power system that is converted to direct-current (DC) power.

The first transistor switch 210 is electrically coupled between the positive power supply node 250A and a first bridge output node 260. The second transistor switch 220 is electrically coupled between the first bridge output node 260 and the negative power supply node 250B. In one example, a source of the first transistor switch 210 is electrically coupled to a drain of the second transistor switch 220 at the first bridge output node 260. The third transistor switch 230 is electrically coupled between the positive power supply node 250A and a second bridge output node 270. The fourth transistor switch 240 is electrically coupled between the second bridge output node 270 and the negative power supply node 250B. In one example, a source of the third transistor switch 230 is electrically coupled to a drain of the fourth transistor switch 240 at the second bridge output node 270.

The bridge circuit 120 also includes a first patient cathode electrode connector 110A and a second patient cathode electrode connector 110B. The first patient cathode electrode connector 110A and the second patient cathode electrode connector 110B are configured to be connected to opposing electrodes (for example, positive and negative electrodes respectively) of the catheter 110 (for example, patient load) to deliver the voltage pulses from the bridge circuit 120. The first patient cathode electrode connector 110A and the second patient cathode electrode connector 110B are electrically coupled between the first bridge output node 260 and the second bridge output node 270. A first relay 280A is provided between the first bridge output node 260 and the first patient cathode electrode connector 110A and a second relay 280B is provided between the second patient cathode electrode connector 110B and the second bridge output node 270. The first relay 280A and the second relay 280B are controlled by the electronic processor 140 to selectively open and close the electrical path between the first bridge output node 260, the catheter 110, and the second bridge output node 270.

The bridge circuit 120 also includes a first patient-isolated internal load connector 290A and a second patient-isolated internal load connector 290B. The first patient-isolated internal load connector 290A and the second patient-isolated internal load connector 290B connect a patient-isolated internal load 290 between the first bridge output node 260 and the second bridge output node 270. The patient-isolated internal load 290 is used for detecting a leakage fault in the bridge circuit 120. A third relay 280C is provided between the first bridge output node 260 and the patient-isolated internal load 290 and a fourth relay 280D is provided between the patient-isolated internal load 290 and the second bridge output node 270. The third relay 280C and the fourth relay 280D are controlled by the electronic processor 140 to selectively open and close the electrical path between the first bridge output node 260, the patient-isolated internal load 290, and the second bridge output node 270.

A first resistor 300A is electrically coupled between (i) the first bridge output node 260 and (ii) the first relay 280A and the third relay 280C. A second resistor 300B is electrically coupled between (i) the second bridge output node 270 and (ii) the second relay 280B and the fourth relay 280D. A third resistor 300C is electrically coupled between the positive power supply node 250A and the first transistor switch 210. A fourth resistor 300D is electrically coupled between the second transistor switch 220 and the negative power supply node 250B. A fifth resistor 300E is electrically coupled between the positive power supply node 250A and the third transistor switch 230. A sixth resistor 300F is electrically coupled between the fourth transistor switch 240 and the negative power supply node 250B. The resistors 300A-F may be used as current detecting elements of the leakage fault protection circuit 130 as described in greater detail below.

The electronic processor 140 is used to control the transistor switches 210-240 and the relays 280 to selectively open and close the electrical paths respectively. A gate driver may be included in the bridge circuit 120 to provide driving signals to the transistor switches 210-240. The gate driver provides driving signals to the transistor switches 210-240 based on the control signals received from the electronic processor 140. When the transistor switches 210-240 are closed, the transistor switches 210-240 allow current to flow through the transistor switches 210-240 to components connected downstream of the transistor switches 210-240. When the transistor switches 210-240 are opened, the transistor switches 210-240 inhibit current flow through the transistor switches 210-240 to components connected downstream of the transistor switches 210-240. Similarly, when the relays 280 are closed, the relays 280 allow current to flow through the relays 280 to components connected downstream of the relays 280. When the relays 280 are opened, the relays 280 inhibit current flow through the relays 280 to components connected downstream of the relays 280.

The first relay 280A and the second relay 280B are closed to form an electrical path between the first bridge output node 260, the catheter 110, and the second bridge output node 270 for delivering therapeutic current to the catheter 110. The third relay 280C and the fourth relay 280D are opened when delivering the therapeutic current to the catheter 110. The electronic processor 140 controls the transistor switches 210-240 to provide sequential bipolar, biphasic high-voltage pulses to the catheter 110. The transistor switches 210-240 may be configured such that the transistor switches 210-240 are normally open. That is, the default state of the transistor switches 210-240 is an open state. The electronic processor 140 closes the first transistor switch 210 and the fourth transistor switch 240 and keeps the second transistor switch 220 and the third transistor switch 230 open to provide therapeutic current in a first direction (for example, a positive direction) to the catheter 110. The electronic processor 140 closes the second transistor switch 220 and the third transistor switch 230 and keeps the first transistor switch 210 and the fourth transistor switch 240 open to provide therapeutic current in a second direction (for example, a negative direction) to the catheter 110. The switching between the first direction and the second direction is performed at a high frequency. For example, the therapeutic current is provided in each direction for 4 microseconds with a 5 microsecond gap between each direction. During the 5 microsecond gap, all transistor switches 210-240 are turned off.

The transistor switches 210-240 are used in the bridge circuit 120 for their near-ideal switch characteristics. Under normal operation, the transistor switches 210-240 allow current to flow through with no voltage drop or very little voltage drop between the drain and the source of the transistor switches 210-240 when closed. The transistor switches 210-240 allow no current or negligible amount of current to flow through the drain and the source of the transistor switches 210-240 when opened. However, the transistor switches 210-240 may sometimes fail and allow leakage current to flow through the transistor switches 210-240 even when the transistor switches 210-240 are opened. This leakage current may cause undesired damage to patient tissues.

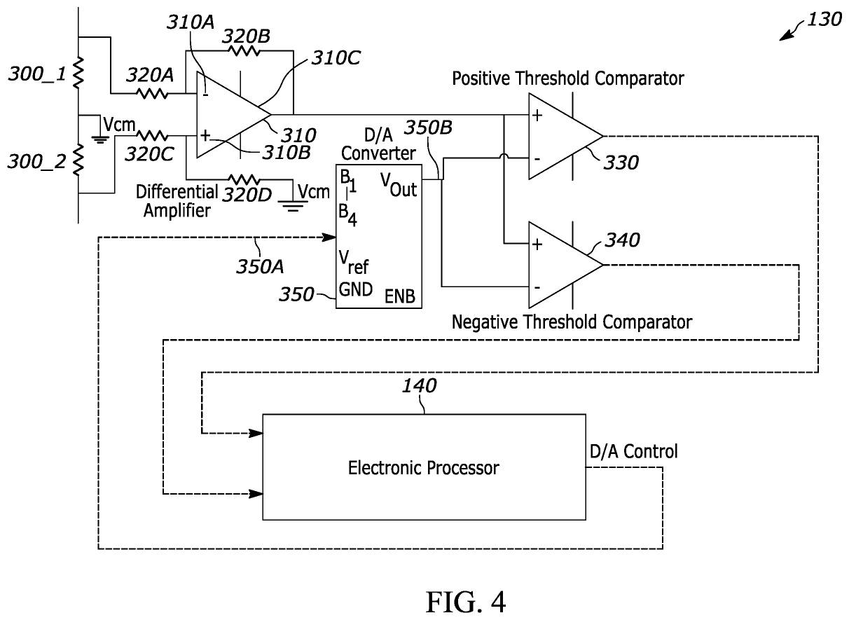

The leakage fault protection circuit 130 may be used during initialization of the pulsed field ablation system 100 to detect a leakage fault. FIG. 4 illustrates a simplified schematic of the leakage fault protection circuit 130. The leakage fault protection circuit 130 may be connected across any one or more of the resistors 300 (for example, current detection elements). In the example illustrated, the leakage fault protection circuit 130 includes series connected measurement resistors 300_1 and 300_2. The measurement resistors 300_1 and 3002 represent any one of the resistors 300. A differential amplifier 310 is connected across the measurement resistors 300_1 and 300_2 such that a first end of the measurement resistors 300_1 and 300_2 is connected to the non-inverting input 310A of the differential amplifier 310 and a second end of the measurement resistors 300_1 and 300_2 is connected to the inverting input 310B of the differential amplifier 310. Resistors 320A-D are connected between the measurement resistors 300_1 and 300_2, the inputs 310A-B of the differential amplifier 310, and an output 310C of the differential amplifier 310 to provide a large voltage gain. The resistors 320A-D may be selected to sufficiently amplify a minimum leakage current (for example, 10 microamperes) to be detected in the pulsed field ablation system 100.

The output 310C of the differential amplifier 310 is electrically coupled to non-inverting inputs of a positive threshold comparator 330 and a negative threshold comparator 340. The leakage fault protection circuit 130 includes a digital to analog converter 350 that receives digital input signals 350A from the electronic processor 140 and provides a threshold parameter 350B to the inverting inputs of the positive threshold comparator 330 and the negative threshold comparator 340. The outputs of the positive threshold comparator 330 and the negative threshold comparator 340 are monitored by the electronic processor 140.

The positive threshold comparator 330 is used to detect a leakage current in the positive direction, for example, when the leakage current is flowing from the first end to the second end of the measurement resistors 300_1 and 300_2. The negative threshold comparator 340 is used to detect a leakage current in the negative direction, for example, when the leakage current is flowing from the second end to the first end of the measurement resistors 300_1 and 300_2. When a current flows across the measurement resistors 300_1 and 300_2, the voltage drop across the measurement resistors 300_1 and 300_2 is amplified by the differential amplifier 310 and a detection parameter proportional to the voltage drop is provided to the non-inverting inputs of the positive threshold comparator 330 and the negative threshold comparator 340. The output of the positive threshold comparator 330 switches states (for example, from high to low or low to high) when the detection parameter at the non-inverting input exceeds the threshold parameter at the inverting input. Similarly, the output of the negative threshold comparator 340 switches states when the detection parameter at the non-inverting input exceeds the threshold parameter at the inverting input. The electronic processor 140 determines the presence of a leakage fault upon detecting the switch in state of the output of the positive threshold comparator 330 or the output of the negative threshold comparator 340.

In some examples, the electronic processor 140 controls the digital to analog converter 350 to provide separate threshold parameters to the positive threshold comparator 330 and the negative threshold comparator 340 to monitor the directionality of the leakage current. For example, the electronic processor 140 controls the digital to analog converter 350 to provide a first threshold parameter at a first time to the positive threshold comparator 330 and the negative threshold comparator 340 and monitors only the output of the positive threshold comparator 330. The electronic processor 140 then controls the digital to analog converter 350 to provide a second threshold parameter at a second time to the positive threshold comparator 330 and the negative threshold comparator 340 and monitors only the output of the negative threshold comparator 340. The first threshold parameter and the second threshold parameter may have different values to account for the directionality of the leakage current flowing across the measurement resistors 300_1 and 300_2. In some instances, the leakage fault protection circuit 130 may use a single threshold comparator to detect leakage fault in either direction.

The differential amplifier 310 can have a small voltage offset between the non-inverting input 310A and the inverting input 310B. This offset varies from part to part and can arise due to manufacturing differences, temperature, offset dependence on supply voltage, or mismatch in sub-components. One example maximum offset specified by manufacturers of differential amplifiers 310 is +/−200 microvolts. Following a large amplifier gain, this offset may rival the detected leakage current and cause a false leakage detection at the positive threshold comparator 330 and/or the negative threshold comparator 340. For example, to accurately detect 10 microamps of leakage through a 70 milliohm resistor (for example, resistor 300) amplified by a differential gain of 800 volt/volt, the positive threshold comparator 330 and the negative threshold comparator 340 should be able to detect 600 microvolts. However, the 200 microvolts offset between the non-inverting input 310A and the inverting input 310B multiplied by the 800 volt/volt gain will yield 160 millivolts at the non-inverting inputs of the positive threshold comparator 330 and the negative threshold comparator 340. Without compensation, the differential amplifier 310 offset may result in false leakage detection at the positive threshold comparator 330 and/or the negative threshold comparator 340. Additionally, the offset referenced at each of the positive threshold comparator 330 and the negative threshold comparator 340 may vary due to nonlinearities in gain, variation in output common mode voltage, and input offset differences between the comparators 330-340.

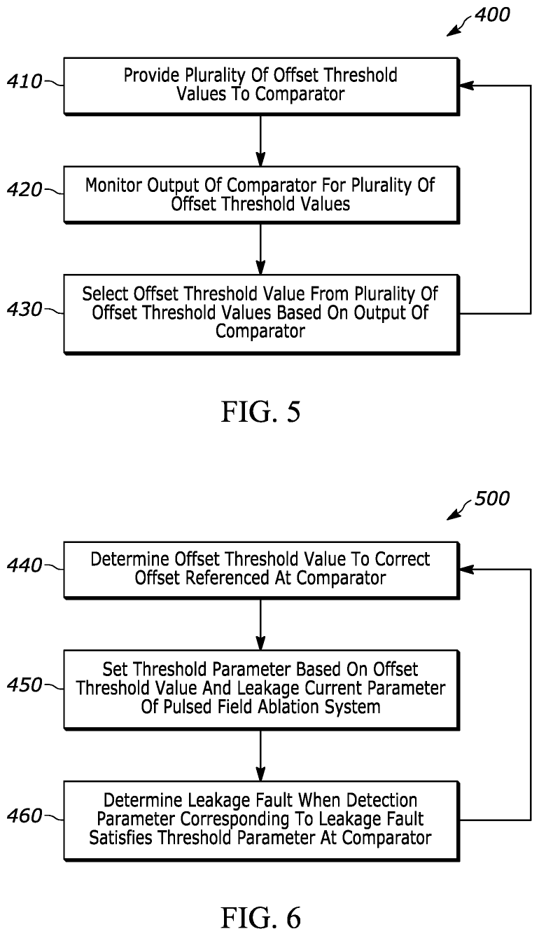

FIG. 5 is a flowchart of an example method 400 for determining an offset threshold value referenced at a comparator 330-340 of the leakage fault protection circuit 130. In the example illustrated, the method 400 includes providing, using the digital to analog converter 350, a plurality of offset threshold values to a comparator 330-340 (for example, a first comparator or a second comparator) (at block 410). The electronic processor 140 controls the digital to analog converter 350 to provide the plurality of offset threshold values. The plurality of offset threshold values may be selected based on the manufacturer specified maximum offset of the differential amplifier 310. Continuing with the example noted above, when the manufacturer specified maximum offset is +/−200 microvolts, the plurality of offset threshold values may be selected to be several discrete values between just below −160 millivolts and just above +160 millivolts (for example, between +/−170 millivolts). In one example, the discrete values may be 10 microvolts apart. The electronic processor 140 may separately select each of the positive threshold comparator 330 and the negative threshold comparator 340 for testing. When the positive threshold comparator 330 is selected, the electronic processor 140 controls the digital to analog converter 350 to scan through the plurality of offset threshold values from the lowest value to the highest value. When the negative threshold comparator 340 is selected, the electronic processor 140 controls the digital to analog converter 350 to scan through the plurality of offset threshold values from the highest value to the lowest value.

The method 400 also includes monitoring, using the electronic processor 140, the output of the comparator 330-340 for the plurality of offset threshold values (at block 420). The electronic processor 140 monitors the outputs of the positive threshold comparator 330 and the negative threshold comparator 340. The outputs of positive threshold comparator 330 and the negative threshold comparator 340 depend on the difference between the inputs of the positive threshold comparator 330 and the negative threshold comparator 340. For example, when the non-inverting input of the positive threshold comparator 330 is below the threshold value provided to the positive threshold comparator 330, the output of the positive threshold comparator 330 is low (for example, −5 Volts). When the non-inverting input of the positive threshold comparator 330 is above the threshold value provided to the positive threshold comparator 330, the output of the positive threshold comparator 330 is high (for example, +5 Volts).

The method 400 includes selecting, using the electronic processor 140, the offset threshold value (for example, a first offset threshold value or a second offset threshold value) from the plurality of offset threshold values based on the output of the comparator 330-340 (at block 430). The electronic processor 140 monitors the outputs of the positive threshold comparator 330 and the negative threshold comparator 340 to determine when the output switches states between high and low. The threshold value from the plurality of offset threshold values at which the output state switches between high and low is determined to be the offset threshold value for the comparator 330-340. The method 400 is repeated for each comparator 330-340, for example, to determine the second offset threshold value for a second comparator.

FIG. 6 is a flowchart of an example method 500 for leakage fault detection in the pulsed field ablation system 100. In the example illustrated, the method 500 includes determining, using the electronic processor 140, the offset threshold value to correct an offset (for example, a first offset or a second offset) referenced at a comparator 330-340 of the leakage fault protection circuit 130 (at block 510). The electronic processor 140 may execute the method 500 for each of the positive threshold comparator 330 and the negative threshold comparator 340 to determine the first offset threshold value and the second offset threshold value.

The method 500 includes setting, using the electronic processor 140, a threshold parameter of the comparator 330-340 based on the offset threshold value and a leakage current parameter of the pulsed field ablation system 100 (at block 520). The leakage current parameter is, for example, a maximum leakage current that can safely be allowed in the pulsed field ablation system 100 or a minimum leakage current to be detected in the pulsed field ablation system 100. In one example, the leakage current parameter is set by the international electrotechnical commission at 10 microamperes under normal conditions and 50 microamperes for a single fault condition. The threshold parameter may be determined by adding the offset threshold value with the product of voltage drop across the resistor 300 due to the leakage current parameter and the differential gain of the leakage fault protection circuit 130. For example, when the offset threshold value is 50 millivolts, the leakage current parameter is 10 microamperes, the resistance value is 75 milliohms, and the differential gain is 800 volt/volt, then the threshold parameter may be set to about 50.6 millivolts (=50 millivolts+(10 microamperes×75 milliohms×800 volt/volt)).

In some instances, the electronic processor 140 determines the offset threshold value over a plurality of iterations (for example, a first plurality of iterations). For example, the electronic processor 140 may determine the offset threshold value over five iterations to compensate for temperature and supply drift during system startup. In these embodiments, the threshold parameter is set when the consecutive offset threshold values converge within, for example, 10 millivolts. The electronic processor 140 may output a fault state when the offset threshold value does not converge or cannot be determined over the plurality of iterations (for example, a second plurality of iterations). For example, the electronic processor 140 may output the fault state when the offset threshold value cannot be determined after twenty iterations. Once the threshold parameters for the positive threshold comparator 330 and the negative threshold comparator 340 are determined, the electronic processor 140 saves the threshold parameters to the memory 150 to be recalled when performing leakage testing.

The method 500 includes determining, using the comparator 330-340, a leakage fault when a detection parameter corresponding to the leakage fault satisfies the threshold parameter at the comparator 330-340 (at block 530). The electronic processor 140 initiates leakage testing once the threshold parameters for the positive threshold comparator 330 and the negative threshold comparator 340 are determined and saved. To perform the leakage testing, the electronic processor 140 closes the relays 280C and 280D to direct any leakage current through the patient-isolated internal load 290 instead of the catheter 110. The electronic processor 140 enables each of the transistor switches 210-240 one after the other for a testing time period (for example, 161 milliseconds each). Specifically, to test one of the transistor switches 210-240, a diagonally opposite transistor switch 210-240 with respect to the patient-isolated internal load 290 is turned on. For example, to test leakage fault in the first transistor switch 210, the electronic processor 140 turns on the fourth transistor switch 240 for 161 milliseconds while turning off the other transistor switches 210-230. The electronic processor 140 monitors the outputs of the positive threshold comparator 330 and the negative threshold comparator 340 to detect a leakage fault in either direction. The leakage test is repeated for each of the transistor switches 210-240. Table 1 below provides an example of the states of the transistor switches 210-240 for testing each of the transistor switches 210-240.

TABLE 1
Transistor Switch States for Leakage Testing

| Test | ON | OFF |
|---|---|---|
| Transistor Switch 210 | Transistor Switch 240 | Transistor Switches |
| | | 210, 220, 230 |
| Transistor Switch 220 | Transistor Switch 230 | Transistor Switches |
| | | 210, 220, 240 |
| Transistor Switch 230 | Transistor Switch 220 | Transistor Switches |
| | | 210, 230, 240 |
| Transistor Switch 240 | Transistor Switch 210 | Transistor Switches |
| | | 220, 230, 240 |

When a leakage fault is detected, the electronic processor 140 may output a fault state providing an indication (for example, turning on an LED, emitting an alarm, or the like) of the fault. In some embodiments, the electronic processor 140 may prevent or inhibit application of current through the catheter 110 when the leakage fault is detected. When no leakage faults are detected, the electronic processor 140 may automatically open the relays 280C and 280D and close the relays 280A and 280B to provide a current path through the catheter 110. The electronic processor 140 then control the transistor switches 210-240 to apply the therapeutic current to the catheter 110.

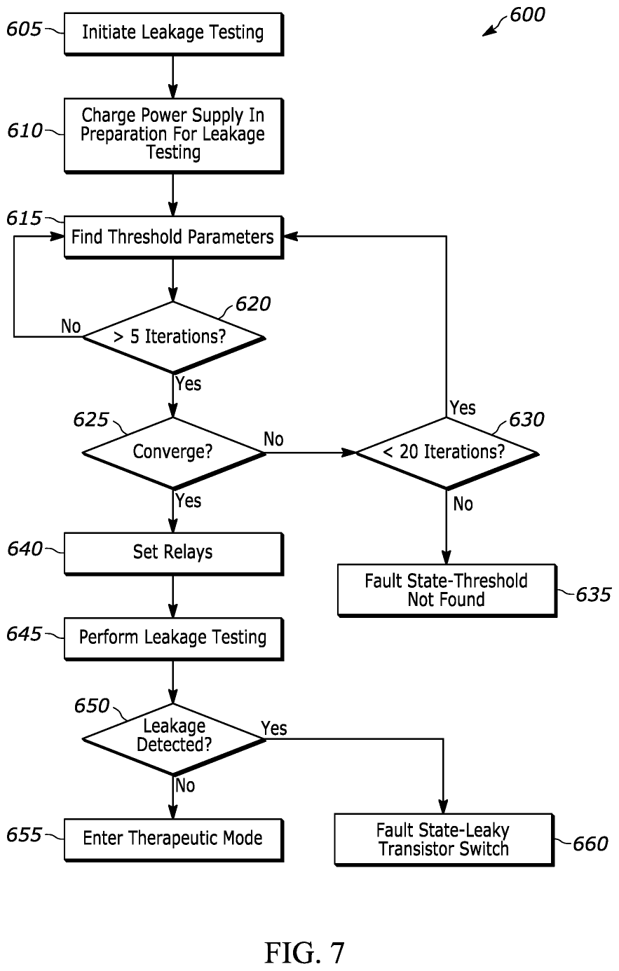

FIG. 7 is a flowchart of an example method 600 for leakage protection in the pulsed field ablation system 100. The flowchart of FIG. 7 illustrates the overall method 600 that may be performed to execute the methods 400 and 500. The method 600 illustrates, for example, a state machine of the pulsed field ablation system 100 for performing leakage protection and may be performed by the electronic processor 140. In the example illustrated, the method 600 includes initiating leakage testing (at block 605). Leakage testing may be initiated at every system startup prior to the pulsed field ablation system being ready for delivering therapeutic current. Initiating leakage testing may include resetting previously determined or stored variables (for example, previously determined threshold parameters or offset threshold values).

The method 600 also includes charging power supply 250 in preparation for leakage system (at block 610). The power supply 250 may be turned on such that the power supply 250 is ready to provide operating power to perform leakage testing. The method 600 includes finding threshold parameters (at block 615). The method 400 may be executed to find the threshold parameters. The method 600 determines whether a minimum number of iterations of block 615 are performed (at block 620) and whether the threshold parameters converge for the minimum number of iterations (at block 625). In on example, the minimum number of iterations is five iterations. In some examples, determining whether the threshold parameters converge includes determining whether the threshold parameters determined for a certain number of consecutive iterations (for example, three consecutive iterations) varies by less than 10 millivolts. When the threshold parameters do not converge for the five iterations, the method 600 includes determining whether a maximum number of iterations of blocks 625 are performed (at block 630). In one example, the maximum number of iterations is twenty iterations. When twenty iterations of block 625 are performed and the threshold parameter does not converge, the method 600 outputs a fault state indicating that a threshold could not be found (at block 635). In response, the electronic processor 140 may provide an indication of the fault state.

When the threshold parameters converge for the five iterations, the method 600 sets the relays 280C and 280D to connect the bridge load (at block 640). The method 600 performs leakage testing (at block 645) for each of the transistor switches 210-240. The method 600 includes determining whether leakage is detected (at block 650) in any of the transistor switches 210-240. When no leakage is detected, the method 600 enters therapeutic mode (at block 655). In the therapeutic mode, the pulsed field ablations system is ready (for example, on standby) to provide therapeutic current through the catheter 110. When a leakage is detected, the method 600 outputs a fault state indicating that a leaky transistor switch 210-240 is found (at block 635). In response, the electronic processor 140 may provide an indication of the fault state.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “electronic processor” as used herein may refer to any of the foregoing structure or any other physical structure suitable for implementation of the described techniques. Also, the techniques could be fully or partially implemented in one or more circuits or logic elements.

The following examples are a non-limiting list of clauses in accordance with one or more techniques of this disclosure.

Example 1. A pulsed field ablation system, comprising: a bridge circuit configured to deliver bipolar and biphasic voltage pulses to a catheter; a leakage fault protection circuit electrically coupled to the bridge circuit and including a first comparator; and an electronic processor electrically coupled to the bridge circuit and the leakage fault protection circuit and configured to determine a first offset threshold value to correct a first offset referenced at the first comparator; set a first threshold parameter of the first comparator based on the first offset threshold value and a leakage current parameter of the pulsed field ablation system; and determine, using the first comparator, a leakage fault when a detection parameter corresponding to the leakage fault satisfies the first threshold parameter at the first comparator.

Example 2. The pulsed field ablation system of Example 1, wherein to determine the first offset threshold value the electronic processor is configured to provide, using a digital to analog converter, a plurality of offset threshold values to the first comparator; monitor an output of the first comparator for the plurality of offset threshold values; and select the first offset threshold value from the plurality of offset threshold values based on the output of the first comparator.

Example 3. The pulsed field ablation system of any of the preceding Examples, wherein the electronic processor is further configured to determine the first offset threshold value over a first plurality of iterations; and set the first threshold parameter when the first offset threshold value converges over the first plurality of iterations.

Example 4. The pulsed field ablation system of any of the preceding Examples, wherein the electronic processor is further configured to determine the first offset threshold value over a second plurality of iterations; and output a fault state when the first offset threshold value does not converge over the second plurality of iterations.

Example 5. The pulsed field ablation system of any of the preceding Examples, wherein the first offset referenced at the first comparator corresponds to an offset between inputs of a differential amplifier of the leakage fault protection circuit.

Example 6. The pulsed field ablation system of any of the preceding Examples, wherein the first comparator is configured to determine a leakage current in a positive direction, wherein the leakage fault protection circuit includes a second comparator configured to determine a leakage current in a negative direction, wherein the electronic processor is configured to determine a second offset threshold value to correct a second offset referenced at the second comparator; set a second threshold parameter of the second comparator based on the second offset threshold value and the leakage current parameter of the pulsed field ablation system; and determine, using the second comparator, the leakage fault in the bridge circuit when the detection parameter corresponding to the leakage fault satisfies the second threshold parameter at the second comparator.

Example 7. The pulsed field ablation system of any of the preceding Examples, wherein the bridge circuit further comprises: a first transistor switch electrically coupled between a positive power supply node and a first bridge output node; a second transistor switch electrically coupled between the first bridge output node and a negative power supply node; a third transistor switch electrically coupled between the positive power supply node and a second bridge output node; and a fourth transistor switch electrically coupled between the second bridge output node and the negative power supply node, wherein the electronic processor is electrically coupled to and controls to selectively open and close the first transistor switch, the second transistor switch, the third transistor switch, and the fourth transistor switch.

Example 8. The pulsed field ablation system of Example 7, wherein the bridge circuit further comprises: a first relay electrically coupling the first bridge output node to a first patient catheter electrode connector; and a second relay electrically coupling the second bridge output node to a second patient catheter electrode connector, a third relay electrically coupling the first bridge output node to a first patient-isolated internal load connector; and a fourth relay electrically coupling the second bridge output node to a second patent-isolated internal load connector, wherein the electronic processor is electrically coupled to the first relay, the second relay, the third relay, and the fourth relay, wherein the electronic processor is configured to close the first relay and the second relay and open the third relay and the fourth relay during therapeutic current delivery; and close the third relay and the fourth relay and open the first relay and the second relay during leakage testing.

Example 9. The pulsed field ablation system of any of Examples 7 and 8, wherein the electronic processor is configured to close the fourth transistor switch and open the first transistor switch, the second transistor switch, and the third transistor switch to determine the leakage fault in the first transistor switch.

Example 10. The pulsed field ablation system of any of Examples 7-9, further comprising: a current detection element is connected between the first bridge output node and the second bridge output node.

Example 11. The pulsed field ablation system of any of the preceding Examples, wherein the leakage fault protection circuit further comprises a differential amplifier connected across a current detection element of the bridge circuit and configured to receive a voltage drop across the current detection element at inputs of the differential amplifier, wherein the first offset is based on an offset between the inputs of the differential amplifier; wherein the first comparator receives an output of the differential amplifier as the detection parameter.

Example 12. The pulsed field ablation system of Example 11, wherein the leakage fault protection circuit further comprises a digital to analog converter connected between the electronic processor and the first comparator, wherein the digital to analog converter is configured to provide the first threshold parameter to the first comparator based on digital inputs received from the electronic processor.

Example 13. The pulsed field ablation system of any of the preceding Examples, wherein the leakage fault protection circuit is configured to detect a minimum leakage current of at least 10 microamperes.

Example 14. A method for leakage fault protection in a pulsed field ablation system including a bridge circuit configured to deliver bipolar and biphasic voltage pulses to a catheter and a leakage fault protection circuit electrically coupled to the bridge circuit and including a first comparator, the method comprising: determining, using an electronic processor, a first offset threshold value to correct a first offset referenced at the first comparator; setting, using the electronic processor, a first threshold parameter of the first comparator based on the first offset threshold value and a leakage current parameter of the pulsed field ablation system; and determining, using the first comparator, a leakage fault when a detection parameter corresponding to the leakage fault satisfies the first threshold parameter at the first comparator.

Example 15. The method of Example 14, wherein to determine the first offset threshold value the method further comprises: providing, using a digital to analog converter, a plurality of offset threshold values to the first comparator; monitoring an output of the first comparator for the plurality of offset threshold values; and selecting the first offset threshold value from the plurality of offset threshold values based on the output of the first comparator.

Example 16. The method of any of Examples 14-15, further comprising: determining the first offset threshold value over a first plurality of iterations; and setting the first threshold parameter when the first offset threshold value converges over the first plurality of iterations.

Example 17. The method of any of Examples 14-16, further comprising: determining the first offset threshold value over a second plurality of iterations; and outputting a fault state when the first offset threshold value does not converge over the second plurality of iterations.

Example 18. The method of any of Examples 14-17, wherein the first offset referenced at the first comparator corresponds to an offset between the inputs of a differential amplifier of the leakage fault protection circuit.

Example 19. The method of any of Examples 14-18, wherein the first comparator is configured to determine a leakage current in a positive direction, wherein the leakage fault protection circuit includes a second comparator configured to determine a leakage current in a negative direction, the method further comprising: determining a second offset threshold value to correct a second offset referenced at the second comparator; setting a second threshold parameter of the second comparator based on the second offset threshold value and the leakage current parameter of the pulsed field ablation system; and determining, using the second comparator, the leakage fault in the bridge circuit when the detection parameter corresponding to the leakage fault satisfies the second threshold parameter at the second comparator.

Example 20. The method of any of Examples 14-19, wherein the leakage fault protection circuit is configured to detect a minimum leakage current of at least 10 microamperes.

1. A pulsed field ablation system, comprising:
a bridge circuit configured to deliver bipolar and biphasic voltage pulses to a catheter;
a leakage fault protection circuit electrically coupled to the bridge circuit and including a first comparator; and
an electronic processor electrically coupled to the bridge circuit and the leakage fault protection circuit and configured to:
determine a first offset threshold value to correct a first offset referenced at the first comparator;
set a first threshold parameter of the first comparator based on the first offset threshold value and a leakage current parameter of the pulsed field ablation system; and
determine, using the first comparator, a leakage fault when a detection parameter corresponding to the leakage fault satisfies the first threshold parameter at the first comparator.

2. The pulsed field ablation system of claim 1, wherein to determine the first offset threshold value the electronic processor is configured to:
provide, using a digital to analog converter, a plurality of offset threshold values to the first comparator;
monitor an output of the first comparator for the plurality of offset threshold values; and
select the first offset threshold value from the plurality of offset threshold values based on the output of the first comparator.

3. The pulsed field ablation system of claim 1, wherein the electronic processor is further configured to:
determine the first offset threshold value over a first plurality of iterations; and
set the first threshold parameter when the first offset threshold value converges over the first plurality of iterations.

4. The pulsed field ablation system of claim 1, wherein the electronic processor is further configured to:
determine the first offset threshold value over a second plurality of iterations; and
output a fault state when the first offset threshold value does not converge over the second plurality of iterations.

5. The pulsed field ablation system of claim 1, wherein the first offset referenced at the first comparator corresponds to an offset between inputs of a differential amplifier of the leakage fault protection circuit.

6. The pulsed field ablation system of claim 1, wherein the first comparator is configured to determine a leakage current in a positive direction, wherein the leakage fault protection circuit includes a second comparator configured to determine a leakage current in a negative direction, wherein the electronic processor is configured to:
determine a second offset threshold value to correct a second offset referenced at the second comparator;
set a second threshold parameter of the second comparator based on the second offset threshold value and the leakage current parameter of the pulsed field ablation system; and
determine, using the second comparator, the leakage fault in the bridge circuit when the detection parameter corresponding to the leakage fault satisfies the second threshold parameter at the second comparator.

7. The pulsed field ablation system of claim 1, wherein the bridge circuit further comprises:
a first transistor switch electrically coupled between a positive power supply node and a first bridge output node;
a second transistor switch electrically coupled between the first bridge output node and a negative power supply node;
a third transistor switch electrically coupled between the positive power supply node and a second bridge output node; and
a fourth transistor switch electrically coupled between the second bridge output node and the negative power supply node,
wherein the electronic processor is electrically coupled to and controls to selectively open and close the first transistor switch, the second transistor switch, the third transistor switch, and the fourth transistor switch.

8. The pulsed field ablation system of claim 7, wherein the bridge circuit further comprises:
a first relay electrically coupling the first bridge output node to a first patient catheter electrode connector;
a second relay electrically coupling the second bridge output node to a second patient catheter electrode connector;
a third relay electrically coupling the first bridge output node to a first patient-isolated internal load connector; and
a fourth relay electrically coupling the second bridge output node to a second patent-isolated internal load connector,
wherein the electronic processor is electrically coupled to the first relay, the second relay, the third relay, and the fourth relay, wherein the electronic processor is configured to:
close the first relay and the second relay and open the third relay and the fourth relay during therapeutic current delivery; and
close the third relay and the fourth relay and open the first relay and the second relay during leakage testing.

9. The pulsed field ablation system of claim 7, wherein the electronic processor is configured to:
close the fourth transistor switch and open the first transistor switch, the second transistor switch, and the third transistor switch to determine the leakage fault in the first transistor switch.

10. The pulsed field ablation system of claim 7, further comprising:
a current detection element is connected between the first bridge output node and the second bridge output node.

11. The pulsed field ablation system of claim 1, wherein the leakage fault protection circuit further comprises a differential amplifier connected across a current detection element of the bridge circuit and configured to receive a voltage drop across the current detection element at inputs of the differential amplifier, wherein the first offset is based on an offset between the inputs of the differential amplifier; wherein the first comparator receives an output of the differential amplifier as the detection parameter.

12. The pulsed field ablation system of claim 11, wherein the leakage fault protection circuit further comprises a digital to analog converter connected between the electronic processor and the first comparator, wherein the digital to analog converter is configured to provide the first threshold parameter to the first comparator based on digital inputs received from the electronic processor.

13. The pulsed field ablation system of claim 1, wherein the leakage fault protection circuit is configured to detect a minimum leakage current of at least 10 microamperes.

14. A method for leakage fault protection in a pulsed field ablation system including a bridge circuit configured to deliver bipolar and biphasic voltage pulses to a catheter and a leakage fault protection circuit electrically coupled to the bridge circuit and including a first comparator, the method comprising:
determining, using an electronic processor, a first offset threshold value to correct a first offset referenced at the first comparator;
setting, using the electronic processor, a first threshold parameter of the first comparator based on the first offset threshold value and a leakage current parameter of the pulsed field ablation system; and
determining, using the first comparator, a leakage fault when a detection parameter corresponding to the leakage fault satisfies the first threshold parameter at the first comparator.

15. The method of claim 14, wherein to determine the first offset threshold value the method further comprises:
providing, using a digital to analog converter, a plurality of offset threshold values to the first comparator;
monitoring an output of the first comparator for the plurality of offset threshold values; and
selecting the first offset threshold value from the plurality of offset threshold values based on the output of the first comparator.
